(19) United States
(12) Patent Application Publication
(10) Pub. No.: US 2011/0097413 A1
(43) Pub. Date: Apr. 28, 2011

(54) SOLID STATE FORMS OF DEFERASIROX SALTS AND PROCESS FOR THE PREPARATION THEREOF

(75) Inventors: Praveen Kumar Neela, Andhra Pradesh (IN); Kishore Charugundla, Andhra Pradesh (IN); Rajendra Suryabhan Patil, Tal-Skahapur (IN); Nitin Sharadchandra Pradhan, Maharashtra (IN); Jon Valgeirsson, Hafnarfjordur (IS)

(73) Assignee: ACTAVIS GROUP PTC EHF, Hafnarfjordur (IS)

(21) Appl. No.: 12/988,836

(22) PCT Filed: Apr. 20, 2009
(86) PCT No.: PCT/IB2009/005783

§ 371 Date: Dec. 13, 2010

(30) Foreign Application Priority Data
Apr. 21, 2008 (IN) 964/CHE/2008

(51) Int. Cl. C07D 249/08; A61K 31/4196; A61K 31/555; A61K 9/12; A61K 9/14; A61P 3/00

(52) U.S. Cl. 424/489; 548/268.6; 548/107; 514/383; 514/184; 424/45

(57) ABSTRACT

Provided herein are novel solid state forms of deferasirox salts, process for the preparation, pharmaceutical compositions, and method of treating thereof. The solid state forms of deferasirox salts are useful for preparing deferasirox (I) in high purity.

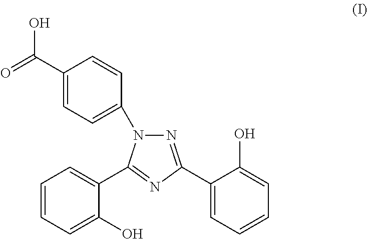

### CROSS REFERENCE TO RELATED APPLICATION

This application claims the benefit of priority to Indian provisional application No. 964/CHE/2008, filed on Apr. 21, 2008, which is incorporated herein by reference in its entirety.

### FIELD OF THE DISCLOSURE

The present disclosure relates to novel solid state forms of deferasirox salts, process for the preparation, pharmaceutical compositions, and method of treating thereof.

### BACKGROUND

U.S. Pat. No. 6,465,504 B1 discloses a variety of substituted 3,5-diphenyl-1,2,4-triazoles, processes for their preparation, pharmaceutical compositions in which they are present and method of use thereof. These compounds are active as iron chelators and useful in the treatment of iron overload in warm-blooded animals. Among them, Deferasirox, 4-[3,5-Bis(2-hydroxyphenyl)-1H-1,2,4-triazol-1-yl]benzoic acid, is an iron chelating agent and it is indicated for the treatment of chronic iron overload due to blood transfusions (transfusional hemosiderosis). Deferasirox is represented by the following structural formula:

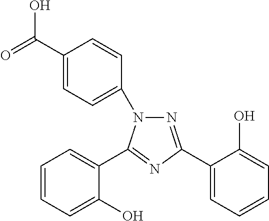

Deferasirox is sold by Novartis under the brand name EXJADE®. Methods of preparing deferasirox are described in U.S. Pat. No. 6,465,504 B1 (herein after referred to as the '504 patent).

The '504 patent describes several synthetic routes for preparing deferasirox. According to one synthetic process, deferasirox is prepared by the ring rearrangement reaction of 1,2,4-dithiazolidine compound of formula III with a substituted hydrazine compound of formula IV in a polar solvent at ambient temperature or elevated temperature up to the reflux temperature of the reaction mixture. However, the experimental details are not provided for this synthetic route.

According to a second synthetic process as described in the '504 patent, deferasirox is prepared by the reaction of 2-(2-hydroxyphenyl)benz[e][1,3]oxazin-4-one with 4-hydrazinobenzoic acid in ethanol at reflux temperature for 2 hours, followed by cooling to precipitate the crystals, and washing with ethanol and then drying to produce deferasirox.

According to a third synthetic process as described in the '504 patent, deferasirox is prepared by the reaction of diacylamine compound of formula VI with a substituted hydrazine compound of formula IV in the presence of polar, protic solvents under weak acid catalysis, preferably in aqueous acetic acid at elevated temperature. However, the experimental details are not provided for this synthetic route.

While the '504 patent mentions that some of the disclosed compounds can form salts with bases, such as appropriate alkali metal or alkaline earth metal salts, e.g. sodium, potassium or magnesium salts; transition metal salts such as zinc salts; or salts with organic amines such as cyclic amines, mono-, di- or tri-lower alkylamines, e.g. ethylamine, tert-butylamine, diethylamine, and diisopropylamine; no salts of the disclosed compounds had been prepared or isolated.

PCT publication No. WO 2008/065123 discloses six crystalline forms including two solvate forms (forms A, B, C, D, S_{A} & S_{B}) and an amorphous form of 4-[3,5-Bis(2-hydroxyphenyl)-[1,2,4]triazol-1-yl]benzoic acid (deferasirox), processes for the preparation, and characterizes them by powder X-ray diffraction (P-XRD), Raman Spectrum and melting points.

U.S. Patent Application No. 2008/0262060 discloses four crystalline forms of deferasirox, methods for the preparation, and pharmaceutical compositions thereof.

There remains a need for novel solid state forms of deferasirox salts.

### SUMMARY

Solid state forms of deferasirox salts have not been reported, isolated, or characterized in the literature. The present inventors have surprisingly and unexpectedly found that salts of 4-[3,5-Bis(2-hydroxyphenyl)-1H-1,2,4-triazol-1-yl]benzoic acid, i.e., deferasirox salts, specifically, triethylamine, dimethylamine, tert-butylamine, sodium (Na⁺), potassium (K⁺), magnesium (Mg²⁺), calcium (Ca²⁺) and zinc (Zn²⁺) salts, can be isolated as solid state forms.

It has also been found that the solid state forms of deferasirox salts are useful intermediates in the preparation of deferasirox or a pharmaceutically acceptable salt thereof in high purity. The solid state forms of deferasirox salts have good flow properties and are far more stable at room temperature, enhanced temperature, at relative high humidities, and in aqueous media. The novel solid state forms of deferasirox salts are suitable for formulating deferasirox.

In one aspect, provided herein are novel solid state forms of a deferasirox salt, wherein the salt is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

In another aspect, deferasirox salts in a crystalline form are provided. In yet another aspect, the crystalline forms of deferasirox salts exist in an anhydrous and/or solvent-free form or as a hydrate and/or a solvate form.

In another aspect, encompassed herein is a process for preparing a solid state form of a deferasirox salt comprising contacting deferasirox free acid with a suitable base in a suitable solvent under suitable conditions to produce a reaction mass, optionally combining the reaction mass with a suitable metal salt, and isolating the appropriate base addition salt of deferasirox as a crystalline form, wherein the base addition salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

In another aspect, provided herein is a method for treating a patient suffering from diseases caused by chronic iron overload due to blood transfusions, comprising administering a solid state form of deferasirox salt, or a pharmaceutical composition that comprises the solid state form of deferasirox salt along with pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

In another aspect, provided herein is a pharmaceutical composition that comprises any one of the solid state forms of deferasirox salts disclosed herein, and one or more pharmaceutically acceptable excipients.

In still another aspect, provided herein is a pharmaceutical composition that comprises any one of the solid state forms of deferasirox salts made by the process disclosed herein, and one or more pharmaceutically acceptable excipients.

In still further aspect, encompassed is a process for preparing a pharmaceutical formulation comprising combining any one of the solid state forms of deferasirox salts disclosed herein with one or more pharmaceutically acceptable excipients.

In another aspect, the solid state forms of deferasirox salts disclosed herein for use in the pharmaceutical compositions has a 90 volume-percent of the particles (D₉₀) having a size of less than or equal to about 500 microns, specifically less than or equal to about 300 microns, more specifically less than or equal to about 100 microns, still more specifically less than or equal to about 60 microns, and most specifically less than or equal to about 15 microns.

### BRIEF DESCRIPTION OF THE DRAWINGS

FIG. 1 is a characteristic powder X-ray diffraction (XRD) pattern of crystalline Deferasirox sodium salt.

FIG. 2 is a characteristic infra red (IR) spectrum of crystalline Deferasirox sodium salt.

FIG. 3 is a characteristic powder X-ray diffraction (XRD) pattern of crystalline Deferasirox potassium salt.

FIG. 4 is a characteristic infra red (IR) spectrum of crystalline Deferasirox potassium salt.

FIG. 5 is a characteristic powder X-ray diffraction (XRD) pattern of crystalline Deferasirox magnesium salt.

FIG. 6 is a characteristic infra red (IR) spectrum of crystalline Deferasirox magnesium salt.

FIG. 7 is a characteristic powder X-ray diffraction (XRD) pattern of crystalline Deferasirox calcium salt.

FIG. 8 is a characteristic infra red (IR) spectrum of crystalline Deferasirox calcium salt.

FIG. 9 is a characteristic powder X-ray diffraction (XRD) pattern of crystalline Deferasirox zinc salt.

FIG. 10 is a characteristic infra red (IR) spectrum of crystalline Deferasirox zinc salt.

FIG. 11 is a characteristic powder X-ray diffraction (XRD) pattern of crystalline Deferasirox triethylamine salt.

FIG. 12 is a characteristic infra red (IR) spectrum of crystalline Deferasirox triethylamine salt.

FIG. 13 is a characteristic powder X-ray diffraction (XRD) pattern of crystalline Deferasirox dimethylamine salt.

FIG. 14 is a characteristic infra red (IR) spectrum of crystalline Deferasirox dimethylamine salt.

FIG. 15 is a characteristic powder X-ray diffraction (XRD) pattern of crystalline Deferasirox tert-butyl amine salt.

FIG. 16 is a characteristic infra red (IR) spectrum of crystalline Deferasirox tert-butylamine salt.

### DETAILED DESCRIPTION

Disclosed herein is the unexpected discovery that deferasirox salts, specifically, triethylamine, dimethylamine, tert-butylamine, sodium (Na⁺), potassium (K⁺), magnesium (Mg²⁺), calcium (Ca²⁺) and zinc (Zn²⁺) salts, can be isolated as solid state forms.

In the formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations (e.g., oral dosage forms such as tablets) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New salt forms of a pharmaceutical agent can further the development of formulations for the treatment of illnesses. For instance, solid forms of a compound are known in the pharmaceutical arts to affect, for example, the solubility, dissolution rate, bioavailability, chemical and physical stability, flowability, fractability, and compressibility of the compound, as well as the safety and efficacy of drug products based on the compound.

The discovery of novel salts in solid state forms of pharmaceutically useful compounds provides a new opportunity to improve the performance characteristics of a pharmaceutical product. It also adds value to the material that a formulation scientist can use the same for designing, for example, a pharmaceutical dosage form of a drug with a targeted release profile or other desired characteristic.

Novel solid state forms of deferasirox salts, specifically, triethylamine, dimethylamine, tert-butylamine, sodium (Na⁺), potassium (K⁺), magnesium (Mg²⁺), calcium (Ca²⁺) and zinc (Zn²⁺) salts have now been discovered.

According to one aspect, provided herein are novel and stable solid state forms of deferasirox salts, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

In one embodiment, the solid state forms of deferasirox salts exist in a crystalline form. In another embodiment, the crystalline forms of deferasirox salts exist in an anhydrous and/or solvent-free form or as a hydrate and/or a solvate form. Such solvated or hydrated forms may be present as hemi-, mono-, sesqui-, di- or tri-solvates or hydrates. Solvates and hydrates may be formed as a result of solvents used during the formation of the deferasirox salts becoming imbedded in the solid lattice structure. Because formation of the solvates and hydrates occurs during the preparation of deferasirox salts, formation of a particular solvated or hydrated form depends greatly on the conditions and method used to prepare the salt. Solvents should be pharmaceutically acceptable.

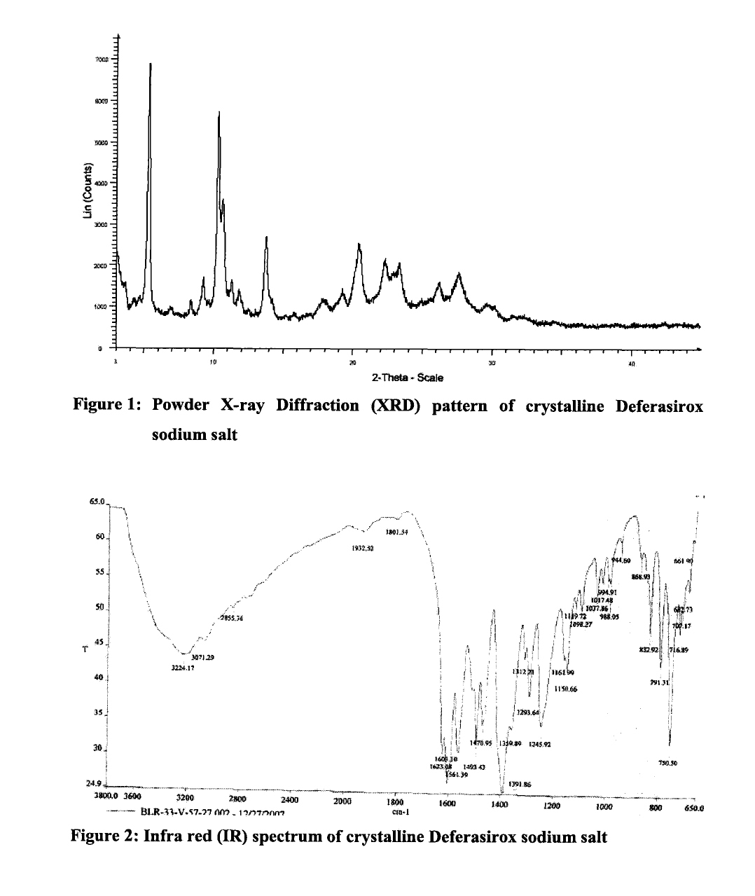

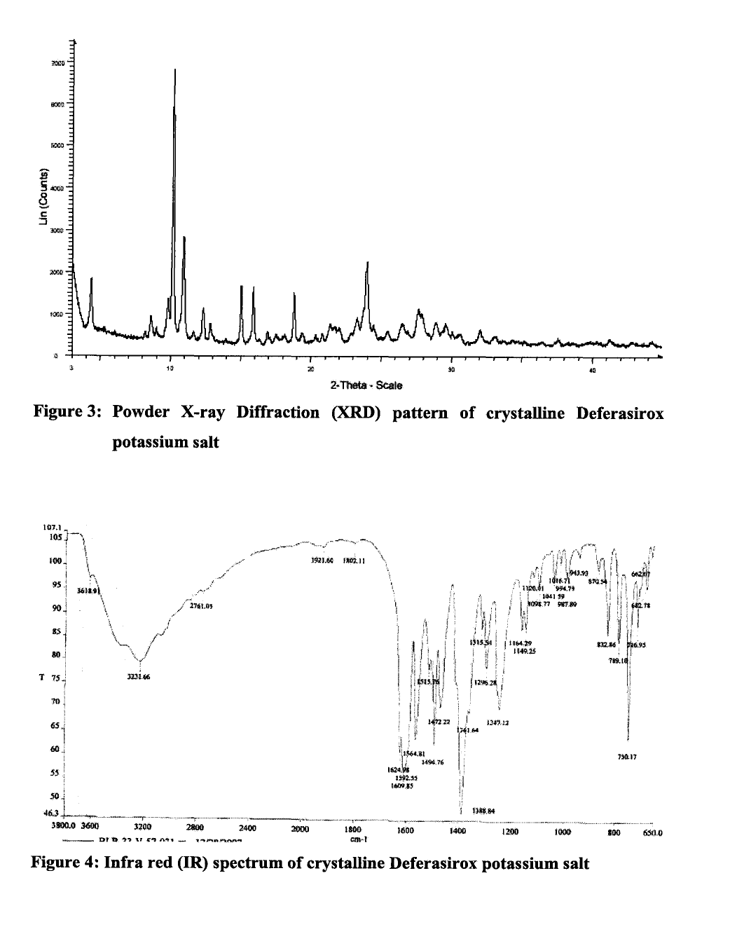

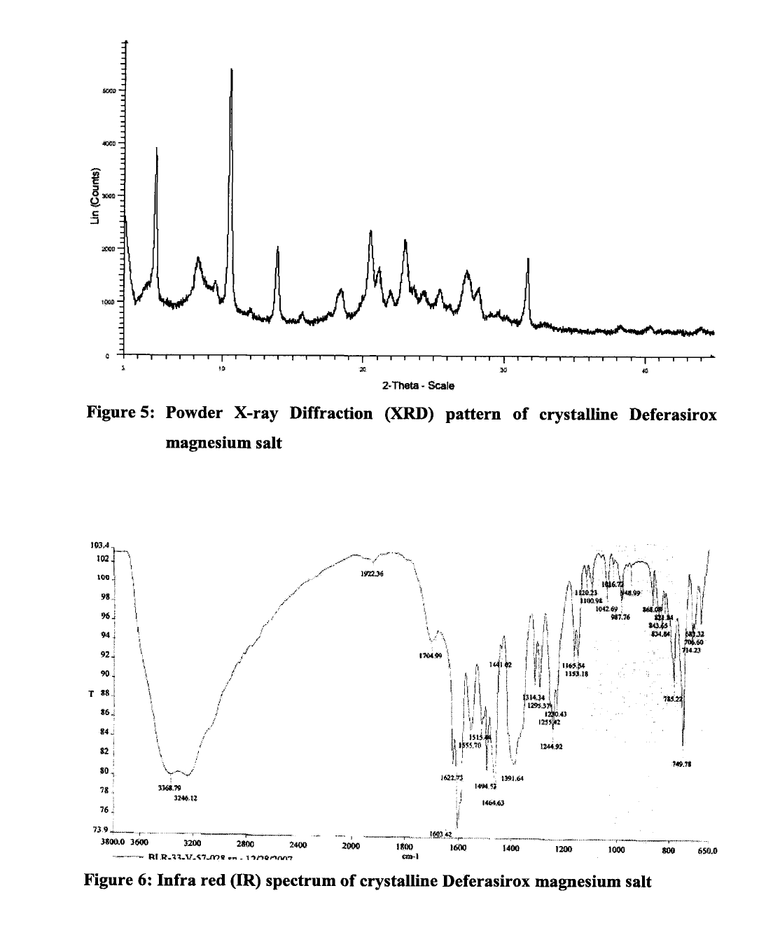

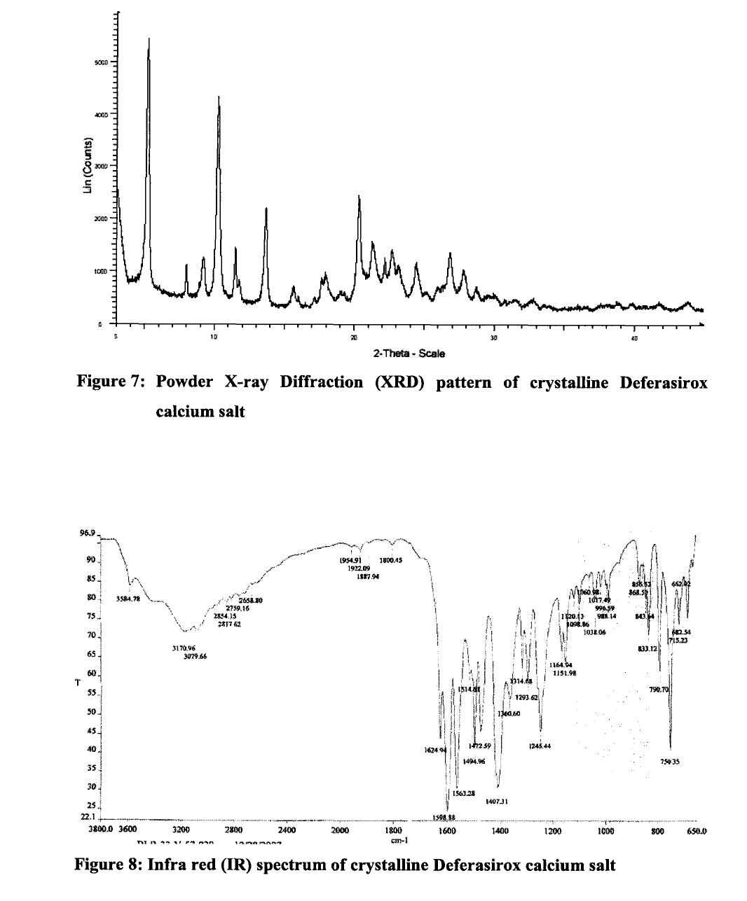

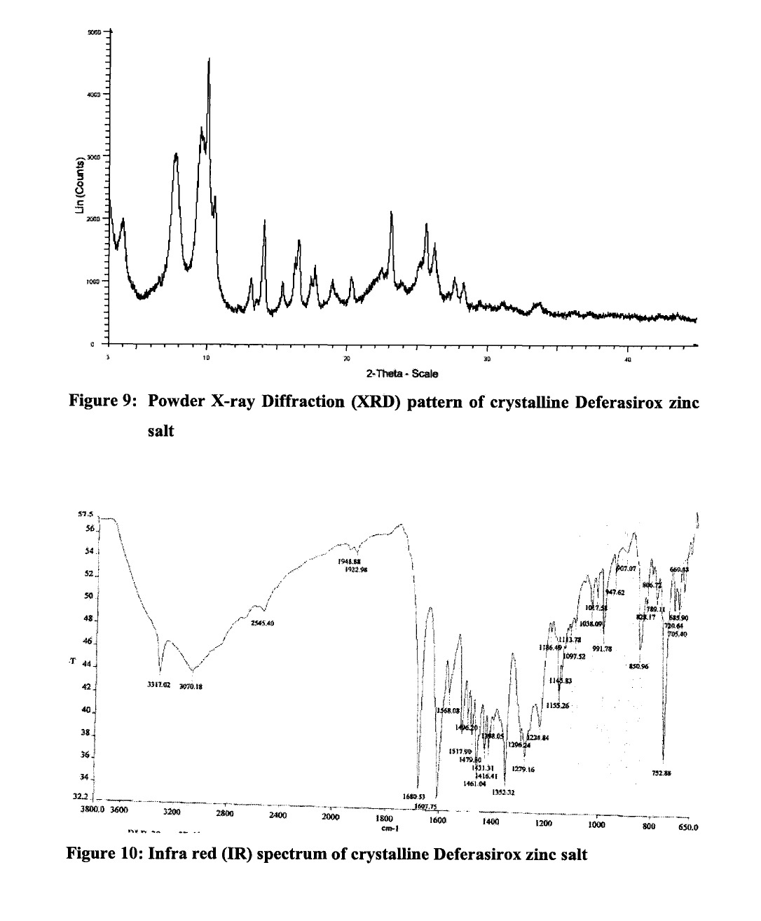

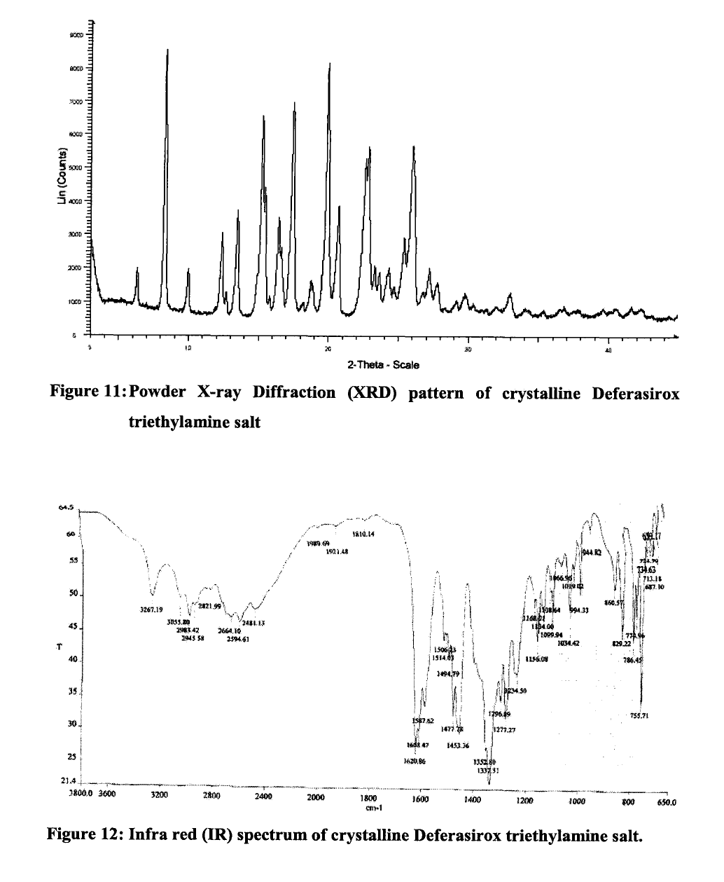

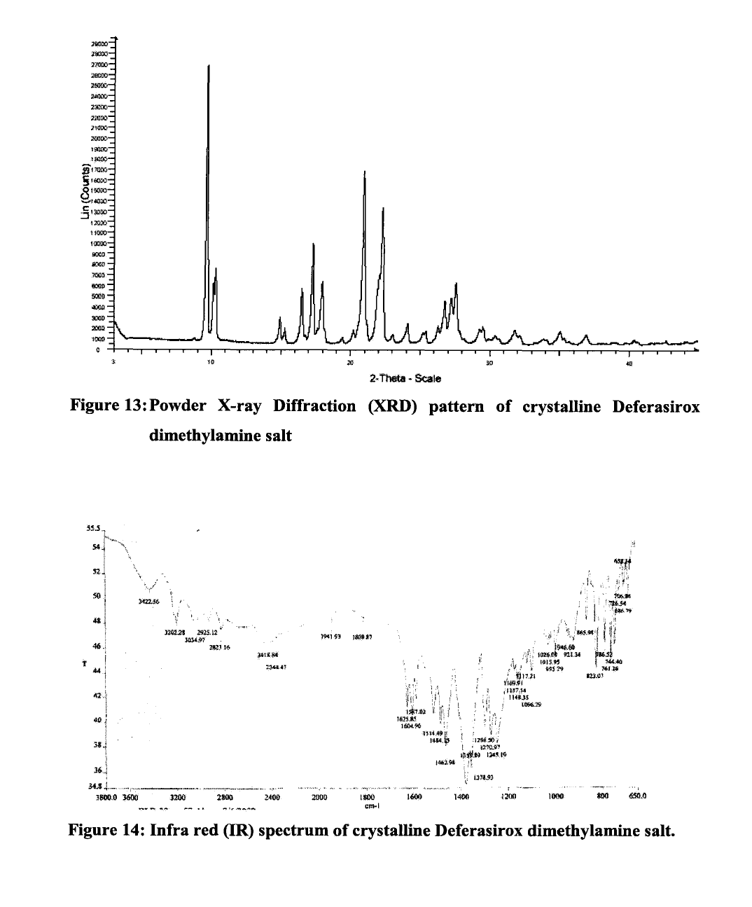

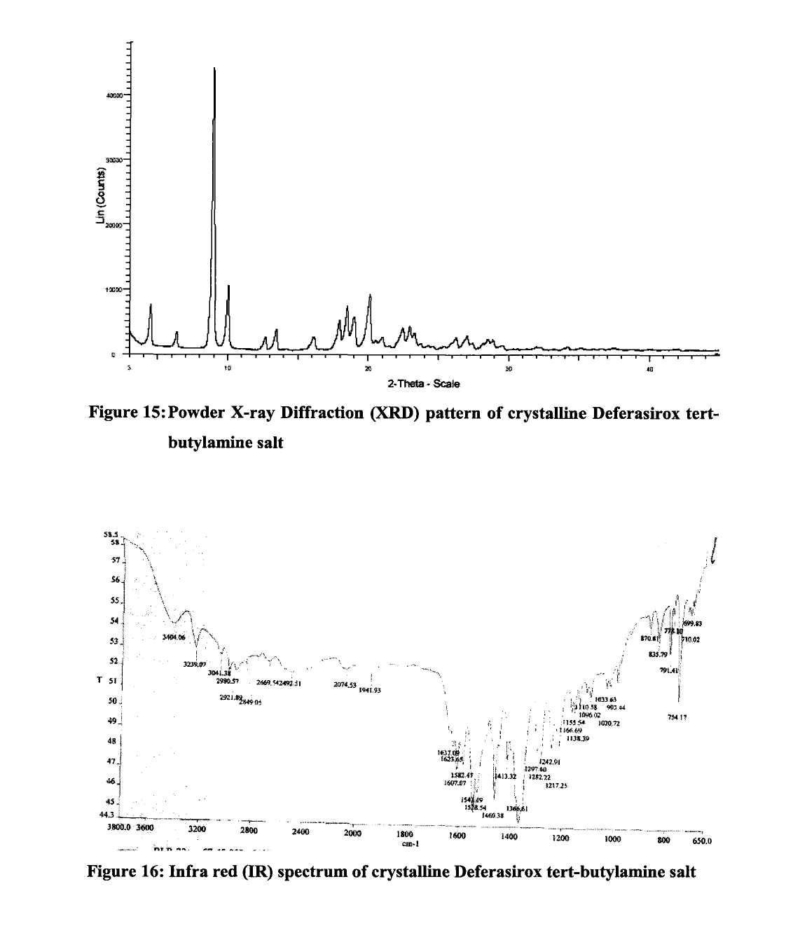

In one embodiment, the solid state forms of deferasirox salts have the following characteristics, wherein:
a) the solid state form of deferasirox sodium salt is characterized by at least one, or more, of the following properties:
   i) a powder X-ray diffraction pattern substantially in accordance with FIG. 1;
   ii) a powder X-ray diffraction pattern having peaks at about 5.27, 10.27, 10.60, 13.71 and 20.40±0.2 degrees 2-theta;
   iii) a powder X-ray diffraction pattern having additional peaks at about 9.21, 11.26, 11.81, 19.24, 22.29, 22.89, 23.32, 26.17 and 27.60±0.2 degrees 2-theta;
   iv) an IR spectrum substantially in accordance with FIG. 2; and
   v) an IR spectrum having absorption bands at about 3224, 1623, 1561, 1470, 1391, 1293, 1245, 1161, 1150, 832, 791 and 750±2 cm⁻¹;
b) the solid state form of deferasirox potassium salt is characterized by at least one, or more, of the following properties:
   i) a powder X-ray diffraction pattern substantially in accordance with FIG. 3;
   ii) a powder X-ray diffraction pattern having peaks at about 4.29, 10.14, 10.89, 15.02, 23.96 and 27.64±0.2 degrees 2-theta;
   iii) a powder X-ray diffraction pattern having additional peaks at about 8.57, 9.79, 12.30, 15.88, 18.81 and 27.88±0.2 degrees 2-theta;
   iv) an IR spectrum substantially in accordance with FIG. 4; and
   v) an IR spectrum having absorption bands at about 3231, 1624, 1609, 1564, 1494, 1472, 1388, 1247, 1164, 1149, 832, 789 and 750±2 cm⁻¹;
c) the solid state form of deferasirox magnesium salt is characterized by at least one, or more, of the following properties
   i) a powder X-ray diffraction pattern substantially in accordance with FIG. 5;
   ii) a powder X-ray diffraction pattern having peaks at about 5.19, 10.49, 13.87, 20.48, 22.96, 27.36 and 31.68±0.2 degrees 2-theta;
   iii) a powder X-ray diffraction pattern having additional peaks at about 8.19, 9.48, 18.43, 21.10 and 28.18±0.2 degrees 2-theta;
   iv) an IR spectrum substantially in accordance with FIG. 6; and
   v) an IR spectrum having absorption bands at about 3368, 3246, 1622, 1603, 1555, 1494, 1464, 1391, 1244, 1165, 1153, 834, 785 and 749±2 cm⁻¹;
d) the solid state form of deferasirox calcium salt is characterized by at least one, or more, of the following properties:
   i) a powder X-ray diffraction pattern substantially in accordance with FIG. 7;
   ii) a powder X-ray diffraction pattern having peaks at about 5.18, 9.18, 13.65, 20.32, 21.33 and 26.85±0.2 degrees 2-theta;
   iii) a powder X-ray diffraction pattern having additional peaks at about 7.98, 10.22, 11.48, 15.65, 17.68, 17.97, 22.18, 22.72, 23.16, 24.43 and 27.84±0.2 degrees 2-theta;
   iv) an IR spectrum substantially in accordance with FIG. 8; and
   v) an IR spectrum having absorption bands at about 3170, 1624, 1598, 1563, 1472, 1407, 1360, 1293, 1245, 1164, 1151, 833, 790 and 750±2 cm⁻¹;
e) the solid state form of deferasirox zinc salt is characterized by at least one, or more, of the following properties:
   i) a powder X-ray diffraction pattern substantially in accordance with FIG. 9;
   ii) a powder X-ray diffraction pattern having peaks at about 7.69, 9.52, 10.0, 10.51, 16.54 and 25.62±0.2 degrees 2-theta;
   iii) a powder X-ray diffraction pattern having additional peaks at about 3.95, 13.13, 14.05, 15.40, 16.30, 17.43, 17.71, 18.95, 20.31, 23.13 and 26.22±0.2 degrees 2-theta;
   iv) an IR spectrum substantially in accordance with FIG. 10; and
   v) an IR spectrum having absorption bands at about 3317, 1680, 1607, 1517, 1479, 1461, 1431, 1416, 1352, 1279, 1224, 991, 850 and 752±2 cm⁻¹;
f) the solid state form of deferasirox triethylamine salt is characterized by at least one, or more, of the following properties:
   i) a powder X-ray diffraction pattern substantially in accordance with FIG. 11;
   ii) a powder X-ray diffraction pattern having peaks at about 8.29, 13.46, 15.24, 15.44, 16.43, 19.92, 20.69, 22.65, 22.82 and 26.03±0.2 degrees 2-theta;
   iii) a powder X-ray diffraction pattern having additional peaks at about 6.28, 9.95, 12.36, 17.45, 18.78, 23.28, 23.63, 24.30, 25.42 and 27.22±0.2 degrees 2-theta;
   iv) an IR spectrum substantially in accordance with FIG. 12; and
   v) an IR spectrum having absorption bands at about 3267, 2983, 1620, 1608, 1587, 1477, 1453, 1352, 1337, 1277, 1234, 1156, 1034, 994, 860, 829, 786 and 755±2 cm⁻¹;
g) the solid state form of deferasirox dimethylamine salt is characterized by at least one, or more, of the following properties:
   i) a powder X-ray diffraction pattern substantially in accordance with FIG. 13;
   ii) a powder X-ray diffraction pattern having peaks at about 9.64, 10.23, 17.28, 17.95, 20.94, 21.97, 22.28 and 27.57±0.2 degrees 2-theta;
   iii) a powder X-ray diffraction pattern having additional peaks at about 14.90, 16.49, 26.76, 27.22 and 27.57±0.2 degrees 2-theta;
   iv) an IR spectrum substantially in accordance with FIG. 14; and
   v) an IR spectrum having absorption bands at about 3422, 3202, 1625, 1604, 1514, 1484, 1462, 1378, 1355, 1296, 1270, 1245, 1117, 823, 786, 761 and 744±2 cm⁻¹;
h) the solid state form of deferasirox tert-butylamine salt is characterized by at least one, or more, of the following properties:
   i) a powder X-ray diffraction pattern substantially in accordance with FIG. 15;
   ii) a powder X-ray diffraction pattern having peaks at about 4.44, 8.91, 9.97, 18.50 and 20.07±0.2 degrees 2-theta;
   iii) a powder X-ray diffraction pattern having additional peaks at about 6.29, 13.41, 17.93 and 18.99±0.2 degrees 2-theta;
   iv) an IR spectrum substantially in accordance with FIG. 16; and
   v) an IR spectrum having absorption bands at about 3404, 3239, 1623, 1607, 1582, 1543, 1528, 1460, 1366, 1297, 1282, 1242, 1217, 1166, 1155, 835, 791 and 754±2 cm⁻¹.

The crystalline forms of deferasirox salts are stable, consistently reproducible, and are particularly suitable for bulk preparation and handling. Moreover, the crystalline forms of deferasirox salts are useful intermediates in the preparation of deferasirox or a pharmaceutically acceptable salt thereof in high purity.

The crystalline forms of deferasirox salts have good flow properties and are far more stable at room temperature, enhanced temperature, at relative high humidities, and in aqueous media. The novel crystalline forms of deferasirox salts are suitable for formulating deferasirox.

According to another aspect, there is provided a process for the preparation of a solid state form of a deferasirox salt, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt; comprising:
a) providing a first solution or a suspension of deferasirox in a first solvent;
b) combining the first solution or suspension with a base to produce a second solution; and
c) if required, substantially removing the first solvent from the second solution to obtain a residue; and
d) dissolving the residue obtained in step-(c) in a second solvent to produce a third solution;
e) if required, combining the deferasirox salt solution obtained in step-(b) or step-(d) with a suitable metal salt to produce a reaction mass; and
f) isolating and/or recovering the crystalline form of deferasirox salt either from the second solution obtained in step-(b) or from the third solution obtained in step-(d) or from the reaction mass obtained in step-(e).

The crystalline forms of deferasirox salts obtained by the process disclosed herein are optionally converted into deferasirox free acid or a pharmaceutically acceptable salt thereof.

The process can produce crystalline forms of deferasirox salts in substantially pure form.

The term “substantially pure crystalline form of deferasirox salt” refers to the crystalline form of deferasirox salt having a purity of greater than about 99 wt %, specifically greater than about 99.5 wt %, more specifically greater than about 99.8 wt %, and still more specifically greater than about 99.9 wt %. The purity can be measured by High Performance Liquid Chromatography (HPLC). For example, the purity of crystalline form of deferasirox salt obtained by the process disclosed herein can be about 99% to about 99.95%, or about 99.5% to about 99.99%, as measured by HPLC.

In one embodiment, the process disclosed herein provides stable crystalline forms of deferasirox salts. The term “stable crystalline form” refers to stability of the crystalline form under the standard temperature and humidity conditions of testing of pharmaceutical products, wherein the stability is indicated by preservation of the original polymorphic form.

Exemplary first and second solvents used in steps-(a) & (d) include, but are not limited to, water, alcohols, ketones, chlorinated hydrocarbons, esters, nitriles, polar aprotic solvents, and mixtures thereof. The term solvent also includes mixtures of solvents.

In one embodiment, the first and second solvents are, each independently, selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, N,N-dimethylformamide, N,N-dimethylacetamide, dimethylsulfoxide, and mixtures thereof

Specifically, the first and second solvents are, each independently, selected from the group consisting of water, methanol, ethanol, isopropyl alcohol, n-butanol, tert-butanol, amyl alcohol, isoamyl alcohol, acetone, and mixtures thereof; and more specifically water, methanol, ethanol, isopropyl alcohol, and mixtures thereof

Step-(a) of providing a first solution of deferasirox includes dissolving deferasirox in the first solvent, or obtaining an existing solution from a previous processing step.

In one embodiment, the deferasirox is dissolved in the first solvent at a temperature of below boiling temperature of the solvent used, specifically at about 25° C. to about 110° C., and more specifically at about 40° C. to about 80° C.

In another embodiment, step-(a) of providing a suspension of deferasirox includes suspending deferasirox in the first solvent while stirring at a temperature below boiling temperature of the solvent used. In one embodiment, the suspension is stirred at a temperature of about 15° C. to about 110° C. for at least 30 minutes and more specifically at a temperature of about 25° C. to about 80° C. for about 1 hour to about 10 hours.

In another embodiment, the solution or suspension in step-(a) is prepared by reacting 2-(2-hydroxyphenyl)-4H-1,3-benzoxazin-4-one with 4-hydrazinobenzoic acid in a reaction inert solvent under suitable conditions to produce a reaction mass containing crude deferasirox, followed by usual work up such as washings, extractions, evaporations, etc. In one embodiment, the work-up includes dissolving, suspending or extracting the resulting deferasirox in the first solvent at a temperature below boiling temperature of the solvent used, specifically at about 25° C. to about 110° C., and more specifically at about 40° C. to about 80° C.

Exemplary reaction inert solvents suitable for facilitating the reaction between 2-(2-hydroxyphenyl)-4H-1,3-benzoxazin-4-one and 4-hydrazinobenzoic acid include, but are not limited to, water, alcohols, ketones, cyclic ethers, aliphatic ethers, hydrocarbons, chlorinated hydrocarbons, nitriles, esters, polar aprotic solvents, and mixtures thereof. In one embodiment, the solvent is selected from the group consisting of water, methanol, ethanol, n-propanol, isopropanol, n-butanol, isobutanol, tert-butanol, amyl alcohol, hexanol, acetone, and mixtures thereof. A specific reaction inert solvent is ethanol.

The first solution or suspension obtained in step-(a) is optionally stirred at a temperature of about 25° C. to the reflux temperature of the solvent used for at least 15 minutes, and specifically at a temperature of about 30° C. to the reflux temperature of the solvent used for about 20 minutes to about 8 hours.

The first solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing deferasirox free acid by removing charcoal or silica gel. Specifically, the finely powdered carbon is an active carbon. A specific mesh size of silica gel is 40-500 mesh, and more specifically 60-120 mesh.

In one embodiment, the base used in step-(b) is an organic or inorganic base. Specific organic bases are triethyl amine, dimethyl amine and tert-butyl amine.

In another embodiment, the base is an inorganic base. In yet another embodiment, the inorganic base is used in the form of an aqueous solution. Exemplary inorganic bases include, but are not limited to, aqueous ammonia; hydroxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate and lithium carbonate, and more specifically sodium hydroxide and potassium hydroxide.

In another embodiment, the base used in step-(b) is in the molar ratio of about 0.8 to 2.5 moles, specifically about 1.0 to 2.0 moles, per mole of deferasirox free acid.

Combining of the first solution or suspension with base in step-(b) is done in a suitable order, for example, the first solution or suspension is added to the base, or alternatively, the base is added to the first solution or suspension. The addition is, for example, carried out drop wise or in one portion or in more than one portion. The addition is specifically carried out at a temperature below about 90° C., more specifically at about 15° C. to about 85° C., and most specifically at about 20° C. to about 80° C. under stirring. After completion of the addition process, the resulting mass is stirred at a temperature of about 30° C. to about 100° C. for at least 10 minutes and specifically at a temperature of about 40° C. to about 80° C. for about 30 minutes to about 8 hours to produce a second solution.

The second solution obtained in step-(b) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing deferasirox salt by removing charcoal or silica gel. Specifically, the finely powdered carbon is an active carbon. A specific mesh size of silica gel is 40-500 mesh, and more specifically 60-120 mesh.

The term “substantially removing” the solvent refers to at least 30%, specifically greater than about 50%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent under inert atmosphere, or a combination thereof, to substantial elimination of total solvent present in the reaction mass.

The distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

The residue containing deferasirox salt in step-(d) is dissolved in the second solvent at a temperature below about reflux temperature of the solvent used, specifically at about 40° C. to about 80° C., and more specifically at about 45° C. to about 60° C.

In one embodiment, the metal salt used in step-(e) is a zinc salt or an alkaline earth metal salt. Specific alkaline earth metals are magnesium and calcium. In yet another embodiment, the metal salt is used in the form of an aqueous solution.

Exemplary metal salts used in step-(e) include, but are not limited to, organic and inorganic salts of magnesium, calcium and zinc, which are capable of dissociating into M²⁺ (wherein M²⁺=Mg²⁺, Ca²⁺ or Zn²⁺), and an anionic component when added to the deferasirox salt solution. Among the organic salts that may be used are carboxylates and sulfonates. Exemplary carboxylates are lower alkyl carboxylates such as acetate, proprionate, butyrate and tartrate; aryl carboxylates such as benzoate and phthalate; and higher alkyl carboxylates such as stearate, dodecanoate and the like. Among the sulfonates that may be used are lower alkyl and aryl sulfonates like calcium methane sulfonate, calcium benzene sulfonate and calcium p-toluene sulfonate.

Exemplary inorganic salts of magnesium, calcium and zinc, include, but are not limited to, halide salts, borates, phosphates and sulfates. Specific inorganic metal salts are halide salts such as magnesium chloride, magnesium bromide, magnesium iodide, calcium chloride, calcium bromide, calcium fluoride, calcium iodide, zinc chloride, zinc bromide and the like; and most specifically, magnesium chloride, calcium chloride and zinc chloride.

In another embodiment, the metal salt used in step-(e) is in the molar ratio of about 0.3 to 1.5 moles, specifically about 0.4 to 0.7 moles, per mole of deferasirox free acid.

Combining of the deferasirox salt solution with metal salt in step-(e) is done in a suitable order, for example, the deferasirox salt solution is added to the metal salt, or alternatively, the metal salt is added to the deferasirox salt solution. The addition is, for example, carried out drop wise or in one portion or in more than one portion. The addition is specifically carried out at a temperature below about 100° C., more specifically at about 30° C. to about 85° C., and most specifically at about 40° C. to about 80° C. under stirring. After completion of the addition process, the resulting mass is stirred at a temperature of about 30° C. to about 100° C. for at least 10 minutes and specifically at a temperature of about 40° C. to about 80° C. for about 30 minutes to about 10 hours.

In one embodiment, the metal salt may be combined with the deferasirox salt solution by adding the metal salt in substantially pure form, i.e., either as a solid or, if liquid, as a neat liquid, to the deferasirox salt solution or, more specifically, by first forming a metal salt solution and then combining the deferasirox salt solution with metal salt solution. In one embodiment, the metal salt and the deferasirox salt solution can be combined by first dissolving the metal salt in a solvent and then adding the metal salt solution to the deferasirox salt solution slowly. Exemplary metal salts are magnesium chloride, calcium chloride or zinc chloride, and the metal salt solvent is water.

The isolation of pure crystalline form of a deferasirox salt in step-(f) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In one embodiment, the crystallization is carried out by cooling the solution at a temperature of below 30° C. for at least 15 minutes, specifically at about 0° C. to about 25° C. for about 30 minutes to about 20 hours, and more specifically at about 0° C. to about 5° C. for about 1 hour to about 8 hours.

The recovering in step-(f) is carried out by methods such as filtration, filtration under vacuum, decantation, centrifugation, or a combination thereof. In one embodiment, crystalline form of deferasirox salt is recovered by filtration employing a filtration media of, for example, a silica gel or celite.

The substantially pure crystalline form of deferasirox salt obtained by the above processes may be further dried in, for example, a Vacuum Tray Dryer, Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the crystalline form of deferasirox salt obtained by the process disclosed herein is greater than about 99%, specifically greater than about 99.5%, more specifically greater than about 99.9%, and most specifically greater than about 99.95% as measured by HPLC. For example, the purity of the crystalline form of deferasirox salt can be about 99% to about 99.95%, or about 99.5% to about 99.99%.

In a specific embodiment, the crystalline deferasirox sodium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution; and
d) isolating crystalline deferasirox sodium from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 1.0 to 2.0 moles, specifically, about 1.0 to 1.5 moles of sodium hydroxide is used per 1 mole of deferasirox free acid.

In another specific embodiment, the crystalline deferasirox potassium salt is prepared by a process comprising:
a) providing a solution or a suspension of deferasirox in a solvent selected from the group consisting of water, alcohols, and mixtures thereof;
b) adding potassium hydroxide to the solution or suspension obtained in step-(a);
c) heating the reaction mass obtained in step-(b) to form a clear solution;
d) optionally, concentrating the solution obtained in step-(c);
e) optionally, dissolving the residue obtained in step-(d) in an alcoholic solvent; and
f) isolating crystalline deferasirox potassium salt from the solution obtained in step-(c) or step-(e).

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 1.0 to 2.0 moles, specifically, about 1.0 to 1.5 moles of potassium hydroxide is used per 1 mole of deferasirox free acid.

In another specific embodiment, the crystalline deferasirox magnesium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution;
d) adding aqueous magnesium chloride solution;
e) optionally, filtering the mass obtained in step-(d) to remove any extraneous matter; and
f) isolating crystalline deferasirox magnesium from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.3 to 0.8 moles, specifically, about 0.4 to 0.7 moles of magnesium chloride is used in step-(d) per 1 mole of deferasirox free acid.

In another specific embodiment, the crystalline deferasirox calcium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution;
d) adding aqueous calcium chloride solution;
e) optionally, filtering the mass obtained in step-(d) to remove any extraneous matter; and
f) isolating crystalline deferasirox calcium from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.3 to 0.8 moles, specifically, about 0.4 to 0.7 moles of calcium chloride is used in step-(d) per 1 mole of deferasirox free acid.

In another specific embodiment, the crystalline deferasirox zinc salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution;
d) adding aqueous zinc chloride solution;
e) optionally, filtering the mass obtained in step-(d) to remove any extraneous matter; and
f) isolating crystalline deferasirox zinc salt from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.3 to 0.8 moles, specifically, about 0.4 to 0.7 moles of zinc chloride is used in step-(d) per 1 mole of deferasirox free acid.

In another specific embodiment, the crystalline deferasirox triethylamine salt is prepared by a process comprising:
a) providing a solution of deferasirox in an alcoholic solvent;
b) adding triethylamine to the solution obtained in step-(a); and
c) isolating crystalline deferasirox triethylamine salt from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.8 to 2.5 moles, specifically, about 1.0 to 2.0 moles of triethylamine is used in step-(b) per 1 mole of deferasirox free acid.

In another specific embodiment, the crystalline deferasirox dimethylamine salt is prepared by a process comprising:
a) providing a solution of deferasirox in an alcoholic solvent;
b) adding dimethylamine to the solution obtained in step-(a); and
c) isolating crystalline deferasirox dimethylamine salt from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.8 to 2.5 moles, specifically, about 1.0 to 2.0 moles of dimethylamine is used in step-(b) per 1 mole of deferasirox free acid.

In another specific embodiment, the crystalline deferasirox tert-butylamine salt is prepared by a process comprising:
a) providing a solution of deferasirox in an alcoholic solvent;
b) adding tert-butylamine to the solution obtained in step-(a); and
c) isolating crystalline deferasirox tert-butylamine salt from the solution.

Specific alcohol solvents are methanol, ethanol, n-propanol, isopropyl alcohol, isobutanol, n-butanol, tert-butanol, amyl alcohol, isoamyl alcohol, and mixtures thereof, and more specifically methanol, ethanol, isopropyl alcohol, and mixtures thereof.

Usually, about 0.8 to 2.5 moles, specifically, about 1.0 to 2.0 moles of tert-butylamine is used in step-(b) per 1 mole of deferasirox free acid.

Deferasirox and pharmaceutically acceptable salts of deferasirox can be prepared in high purity by using the substantially pure crystalline form of deferasirox salt obtained according to the process disclosed herein.

Further encompassed herein is the use of the solid state form of deferasirox salt, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt, for the manufacture of a pharmaceutical composition together with a pharmaceutically acceptable carrier.

A specific pharmaceutical composition of the solid state form of deferasirox salt is selected from a solid dosage form and an oral suspension.

In one embodiment, the solid state form of deferasirox salt, wherein the salt of deferasirox is a triethylamine salt, a dimethylamine salt, a tert-butylamine salt, a sodium (Na⁺) salt, a potassium (K³⁰ ) salt, a magnesium (Mg²⁺) salt, a calcium (Ca²⁺) salt or a zinc (Zn²⁺) salt, has a D₉₀ particle size of less than or equal to about 500 microns, specifically less than or equal to about 300 microns, more specifically less than or equal to about 100 microns, still more specifically less than or equal to about 60 microns, and most specifically less than or equal to about 15 microns.

In another embodiment, the particle sizes of the solid state form of deferasirox salt are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising the solid state form of deferasirox salt and one or more pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

According to another aspect, there is provided pharmaceutical compositions comprising the solid state form of deferasirox salt prepared according to process disclosed herein and one or more pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

According to another aspect, there is provided a process for preparing a pharmaceutical formulation comprising combining the solid state form of deferasirox salt prepared according to processes disclosed herein, with one or more pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

According to another aspect, there is provided a method for treating a patient suffering from diseases caused by chronic iron overload due to blood transfusions, comprising administering the solid state form of deferasirox salt, or a pharmaceutical composition that comprises the solid state form of deferasirox salt along with pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

Yet in another embodiment, pharmaceutical compositions comprise at least a therapeutically effective amount of solid state form of a deferasirox salt, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The solid state form of deferasirox salt may also be administered as suppositories, ophthalmic ointments and suspensions, and parenteral suspensions, which are administered by other routes, wherein the salt of deferasirox is a triethylamine salt, a dimethylamine salt, a tert-butylamine salt, a sodium (Na⁺) salt, a potassium (K⁺) salt, a magnesium (Mg²) salt, a calcium (Ca²⁺) salt or a zinc (Zn²⁺) salt.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain crystalline form of deferasirox salt within a capsule which may be coated with gelatin. Tablets and powders may also be coated with an enteric coating. Suitable enteric coating include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-45° 2-theta; step width=0.01579°; and measuring time per step=0.11 sec.

### Infra-Red Spectroscopy (FT-IR):

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

The following examples are given for the purpose of illustrating the present disclosure and should not be considered as limitation on the scope or spirit of the disclosure.

### EXAMPLES

### Example 1

### Preparation of 2-(2-Hydroxyphenyl)-4H-1,3-benzoxazine-4-one

Salicylic acid (50 gm) was taken in xylene (250 ml) followed by the addition of thionyl chloride (64.5 gm) drop wise to the reaction mixture at 25-30° C. The reaction mixture was stirred for 90 minutes at 40-45° C. The excess thionyl chloride was removed by distillation. Salicylamide (49.7 gm) was added to the resulting mixture and followed by the distillation of xylene up to a reaction temperature of 170° C. The reaction mixture was further stirred for 60 minutes at 80° C. followed by the addition of ethanol (80 ml) and refluxed for 15 minutes. The resulting mass was cooled to 25° C. and stirred for 30 minutes at the same temperature. The resulting solid was filtered and dried to produce 43 gm of 2-(2-hydroxyphenyl)-4H-1,3-benzoxazine-4-one as slightly yellow crystals. (Melting point: 206-208° C.).

### Example 2

### Preparation of 2-(2-Hydroxyphenyl)-4H-1,3-benzoxazine-4-one

Salicylic acid (50 gm) was taken in toluene (125 ml) followed by the addition of thionyl chloride (64.5 gm) drop wise to the reaction mixture at 20-30° C. The reaction mixture was stirred for 90 minutes at 40-45° C. The resulting mass was distilled under vacuum until to remove about 100 ml of toluene along with the excess thionyl chloride. Salicylamide (49.7 gm) was added to the resulting mixture and followed by the distillation of the toluene up to a reaction temperature of 170° C. The reaction mixture was further stirred for 60 minutes at 165-170° C. followed by the addition of methanol (150 ml) at 60° C. and refluxed for 15 minutes. The resulting mass was cooled to 25° C. and stirred for 30 minutes at the same temperature. The resulting solid was filtered and dried to produce 43 gm of 2-(2-hydroxyphenyl)-4H-1,3-benzoxazine-4-one as slightly yellow crystals. (Melding point: 206-208° C.).

### Example 3

### Preparation of 4-[3,5-Bis(2-hydroxyphenyl)-1H-1,2,4-triazol-1-yl]benzoic acid (Deferasirox)

2-(2-Hydroxyphenyl)-4H-1,3-benzoxazine-4-one (25 gm, 0.1045 moles) and 4-hydrazinobenzoic acid (17.5 gm, 0.115 moles) were taken in ethanol (375 ml). The reaction mass was heated to reflux and refluxed for 2 hours. The resulting mass was cooled to 25° C., the resulting solid was filtered and dried to produce 30.4 gm of 4-[3,5-Bis(2-hydroxyphenyl)-1H-1,2,4-triazol-1-yl]benzoic acid (Melting point: 264° C.).

### Example 4

### Preparation of Deferasirox Sodium Salt

4-[3,5-Bis(2-hydroxyphenyl)-1H-1,2,4-triazol-1-yl]benzoic acid (1 gm) was suspended in water (5 ml). This was followed by the drop wise addition of a solution of sodium hydroxide (0.117 gm) in water (2 ml) at 22-25° C. The reaction mixture was heated at 50-55° C. to form a clear solution. The resulting solution was concentrated under vacuum. This was followed by the addition of water (3 ml) and heating of the resulting mass to get a clear solution which was further cooled at 0° C. The resulting solid was filtered and further dried under vacuum at 80-90° C. to produce 0.65 gm of crystalline deferasirox sodium salt (Purity by HPLC: 99.99%).

### Example 5

### Preparation of Deferasirox Potassium Salt

4-[3,5-Bis(2-hydroxyphenyl)-1H-1,2,4-triazol-1-yl]benzoic acid (2 gm) was suspended in water (15 ml) at 22-25° C. This was followed by the drop wise addition of a solution of potassium hydroxide (0.33 gm) in water (2 ml) at 22-25° C. The resulting mass was heated at 50-55° C. to get a clear solution. The resulting solution was concentrated under vacuum and followed by the addition of isopropyl alcohol (20 ml). The reaction mixture was further heated at 55° C. for 30 minutes and then cooled at 0° C. The resulting solid was filtered and further dried under vacuum at 80-90° C. to produce 1.4 gm of crystalline deferasirox potassium salt (Purity by HPLC: 99.97%).

### Example 6

### Preparation of Deferasirox Magnesium Salt

### Method-I:

4-[3,5-Bis(2-hydroxyphenyl)-1H-1,2,4-triazol-1-yl]benzoic acid (2 gm) was suspended in water (10 ml) at 22-25° C. and followed by the drop wise addition of a solution of sodium hydroxide (0.235 gm) in water (4 ml) at 22-25° C. The resulting mass was heated at 50-55° C. to get a clear solution. This was followed by the slow addition of a solution of magnesium chloride (0.55 gm) in water (6 ml). The precipitated product was further stirred for 1 hour at 25-30° C. The resulting solid was filtered and further dried under vacuum at 80-90° C. to produce 1.2 gm of crystalline deferasirox magnesium salt (Purity by HPLC: 99.76%).

### Method-II:

4-[3,5-Bis(2-hydroxyphenyl)-1H-1,2,4-triazol-1-yl]benzoic acid (1 gm) was suspended in methanol (10 ml) at 22-25° C. This was followed by the drop wise addition of a solution of sodium hydroxide (0.117 gm) in methanol (5 ml) at 22-25° C. The reaction mixture was heated at 50-55° C. to get a clear solution and followed by the addition of a solution of magnesium chloride (0.279 gm) in water (1 ml). The resulting solid was filtered and further dried under vacuum at 65-70° C. to produce 0.5 gm of crystalline deferasirox magnesium salt (Purity by HPLC: 99.81%).

### Example 7

### Preparation of Deferasirox Calcium Salt

4-[3,5-Bis(2-hydroxyphenyl)-1H-1,2,4-triazol-1-yl]benzoic acid (2 gm) was suspended in water (5 ml) at 22-25° C. and followed by the drop wise addition of a solution of sodium hydroxide (0.235 gm) in water (5 ml) at 22-25° C. The resulted mass was heated at temperature 50-55° C. to get a clear solution. This was followed by the addition of a solution of calcium chloride (0.654 gm) in water (6 ml) and the resulting solid was filtered and further dried under vacuum at 80-90° C. to produce 1.27 gm of crystalline deferasirox calcium salt (Purity by HPLC: 99.97%).

### Example 8

### Preparation of Deferasirox Zinc Salt

4-[3,5-Bis(2-hydroxyphenyl)-1H-1,2,4-triazol-1-yl]benzoic acid (2 gm) was suspended in water (20 ml) at 22-25° C. and followed by the drop wise addition of a solution of sodium hydroxide (0.235 gm) in water (5 ml) at 22-25° C. The reaction mass was heated at temperature 50-55° C. to get a clear solution. This was followed by the addition of a solution of zinc chloride (0.801 gm) in water (4 ml) and the resulting solid was filtered and further dried under vacuum at 80-90° C. to produce 2 gm of crystalline deferasirox zinc salt (Purity by HPLC: 99.94%).

### Example 9

### Preparation of Triethylamine Salt of Deferasirox

4-[3,5-Bis(2-hydroxyphenyl)-1H-1,2,4-triazol-1-yl]benzoic acid (1 gm) was suspended in isopropyl alcohol (40 ml) at 22-25° C. and refluxed at 78-80° C. to get a clear solution. This was followed by the drop wise addition of triethylamine (0.41 ml) at 78-80° C. The resulting mass was cooled at 25° C. and the resulting solid was filtered and further dried under vacuum at 70-80° C. to produce 1.22 gm of crystalline deferasirox triethylamine salt (Purity by HPLC: 99.97%).

### Example 10

### Preparation of tert-butylamine Salt of Deferasirox

4-[3,5-Bis(2-hydroxyphenyl)-1H-1,2,4-triazol-1-yl]benzoic acid (2 gm) was suspended in isopropyl alcohol (80 ml) and heated at 78-80° C. to get a clear solution. This was followed by the drop wise addition of tert-butylamine (0.564 ml) at reflux temperature. The reaction mixture was cooled at 25° C. and the resulting solid was filtered and further dried under vacuum at 70-80° C. to produce 0.7 gm of crystalline deferasirox tert-butylamine salt (Purity by HPLC: 99.97%).

### Example 11

### Preparation of Dimethylamine Salt of Deferasirox

4-[3,5-Bis(2-hydroxyphenyl)-1H-1,2,4-triazol-1-yl]benzoic acid (2 gm) was suspended in isopropyl alcohol (80 ml) at 22-25° C. and further refluxed at 78-80° C. to get a clear solution. This was followed by the drop wise addition of dimethylamine (0.679 ml) at 78-80° C. The reaction mixture was then cooled at 25° C. and the resulting solid was filtered and further dried under vacuum at 70-80° C. to produce 2 gm of crystalline deferasirox dimethylamine salt (Purity by HPLC: 99.98%).

Unless otherwise indicated, the following definitions are set forth to illustrate and define the meaning and scope of the various terms used to describe the invention herein.

The term “solid state form of deferasirox salt disclosed herein” includes crystalline forms, amorphous forms, hydrated, and solvated forms of deferasirox salt.

The term “crystalline form” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

As used herein, D_{X} means that X percent of the particles have a diameter less than a specified diameter D. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Solid state form of a salt of 4-[3,5-Bis(2-hydroxyphenyl)-1H-1,2,4-triazol-1-yl]benzoic acid (deferasirox salt), wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

2. The solid state form of deferasirox salt of claim 1, which is in a crystalline form, and wherein the solid state form is anhydrous and/or solvent-free form or a hydrate and/or a solvate form.

3. (canceled)

4. The solid state form of deferasirox salt of claim 1, having the following characteristics, wherein:
a) the solid state form of deferasirox sodium salt is characterized by one or more of the following properties:
i) a powder X-ray diffraction pattern substantially in accordance with FIG. 1;
ii) a powder X-ray diffraction pattern having peaks at about 5.27, 10.27, 10.60, 13.71 and 20.40±0.2 degrees 2-theta;
iii) a powder X-ray diffraction pattern having additional peaks at about 9.21, 11.26, 11.81, 19.24, 22.29, 22.89, 23.32, 26.17 and 27.60±0.2 degrees 2-theta;
iv) an IR spectrum substantially in accordance with FIG. 2; and
v) an IR spectrum having absorption bands at about 3224, 1623, 1561, 1470, 1391, 1293, 1245, 1161, 1150, 832, 791 and 750±2 cm⁻¹;
b) the solid state form of deferasirox potassium salt is characterized by one or more of the following properties:
i) a powder X-ray diffraction pattern substantially in accordance with FIG. 3;
ii) a powder X-ray diffraction pattern having peaks at about 4.29, 10.14, 10.89, 15.02, 23.96 and 27.64±0.2 degrees 2-theta;
iii) a powder X-ray diffraction pattern having additional peaks at about 8.57, 9.79, 12.30, 15.88, 18.81 and 27.88±0.2 degrees 2-theta;
iv) an IR spectrum substantially in accordance with FIG. 4; and
v) an IR spectrum having absorption bands at about 3231, 1624, 1609, 1564, 1494, 1472, 1388, 1247, 1164, 1149, 832, 789 and 750±2 cm⁻¹;
c) the solid state form of deferasirox magnesium salt is characterized by one or more of the following properties
i) a powder X-ray diffraction pattern substantially in accordance with FIG. 5;
ii) a powder X-ray diffraction pattern having peaks at about 5.19, 10.49, 13.87, 20.48, 22.96, 27.36 and 31.68±0.2 degrees 2-theta;
iii) a powder X-ray diffraction pattern having additional peaks at about 8.19, 9.48, 18.43, 21.10 and 28.18±0.2 degrees 2-theta;
iv) an IR spectrum substantially in accordance with FIG. 6; and
v) an IR spectrum having absorption bands at about 3368, 3246, 1622, 1603, 1555, 1494, 1464, 1391, 1244, 1165, 1153, 834, 785 and 749±2 cm⁻¹;
d) the solid state form of deferasirox calcium salt is characterized by one or more of the following properties:
i) a powder X-ray diffraction pattern substantially in accordance with FIG. 7;
ii) a powder X-ray diffraction pattern having peaks at about 5.18, 9.18, 13.65, 20.32, 21.33 and 26.85±0.2 degrees 2-theta;
iii) a powder X-ray diffraction pattern having additional peaks at about 7.98, 10.22, 11.48, 15.65, 17.68, 17.97, 22.18, 22.72, 23.16, 24.43 and 27.84±0.2 degrees 2-theta;
iv) an IR spectrum substantially in accordance with FIG. 8; and
v) an IR spectrum having absorption bands at about 3170, 1624, 1598, 1563, 1472, 1407, 1360, 1293, 1245, 1164, 1151, 833, 790 and 750±2 cm⁻¹;
e) the solid state form of deferasirox zinc salt is characterized by one or more of the following properties:
i) a powder X-ray diffraction pattern substantially in accordance with FIG. 9;
ii) a powder X-ray diffraction pattern having peaks at about 7.69, 9.52, 10.0, 10.51, 16.54 and 25.62±0.2 degrees 2-theta;
iii) a powder X-ray diffraction pattern having additional peaks at about 3.95, 13.13, 14.05, 15.40, 16.30, 17.43, 17.71, 18.95, 20.31, 23.13 and 26.22±0.2 degrees 2-theta;
iv) an IR spectrum substantially in accordance with FIG. 10; and
v) an IR spectrum having absorption bands at about 3317, 1680, 1607, 1517, 1479, 1461, 1431, 1416, 1352, 1279, 1224, 991, 850 and 752±2 cm⁻¹;
f) the solid state form of deferasirox triethylamine salt is characterized by one or more of the following properties:
i) a powder X-ray diffraction pattern substantially in accordance with FIG. 11;
ii) a powder X-ray diffraction pattern having peaks at about 8.29, 13.46, 5.24, 15.44, 16.43, 19.92, 20.69, 22.65, 22.82 and 26.03±0.2 degrees 2-theta;
iii) a powder X-ray diffraction pattern having additional peaks at about 6.28, 9.95, 12.36, 17.45, 18.78, 23.28, 23.63, 24.30, 25.42 and 27.22±0.2 degrees 2-theta;
iv) an IR spectrum substantially in accordance with FIG. 12; and
v) an IR spectrum having absorption bands at about 3267, 2983, 1620, 1608, 1587, 1477, 1453, 1352, 1337, 1277, 1234, 1156, 1034, 994, 860, 829, 786 and 755±2 cm⁻¹;
g) the solid state form of deferasirox dimethylamine salt is characterized by one or more of the following properties:
i) a powder X-ray diffraction pattern substantially in accordance with FIG. 13;
ii) a powder X-ray diffraction pattern having peaks at about 9.64, 10.23, 17.28, 17.95, 20.94, 21.97, 22.28 and 27.57±0.2 degrees 2-theta;
iii) a powder X-ray diffraction pattern having additional peaks at about 14.90, 16.49, 26.76, 27.22 and 27.57±0.2 degrees 2-theta;
iv) an IR spectrum substantially in accordance with FIG. 14; and
v) an IR spectrum having absorption bands at about 3422, 3202, 1625, 1604, 1514, 1484, 1462, 1378, 1355, 1296, 1270, 1245, 1117, 823, 786, 761 and 744±2 cm⁻¹;
h) the solid state form of deferasirox tert-butylamine salt is characterized by one or more of the following properties:
i) a powder X-ray diffraction pattern substantially in accordance with FIG. 15;
ii) a powder X-ray diffraction pattern having peaks at about 4.44, 8.91, 9.97, 18.50 and 20.07±0.2 degrees 2-theta;
iii) a powder X-ray diffraction pattern having additional peaks at about 6.29, 13.41, 17.93 and 18.99±0.2 degrees 2-theta;
iv) an IR spectrum substantially in accordance with FIG. 16; and
v) an IR spectrum having absorption bands at about 3404, 3239, 1623, 1607, 1582, 1543, 1528, 1460, 1366, 1297, 1282, 1242, 1217, 1166, 1155, 835, 791 and 754±2 cm⁻¹.

5. A process for the preparation of solid deferasirox salt of claim 1, comprising:
a) providing a first solution or a suspension of deferasirox in a first solvent;
b) combining the first solution or suspension with a base to produce a second solution, wherein the base is selected from the group consisting of triethyl amine, dimethyl amine, tert-butyl amine, aqueous ammonia, sodium hydroxide, calcium hydroxide, magnesium hydroxide, potassium hydroxide, lithium hydroxide, sodium carbonate, potassium carbonate and lithium carbonate; and
c) optionally, substantially removing the first solvent from the second solution to obtain a residue; and
d) dissolving the residue obtained in step-(c) in a second solvent to produce a third solution;
e) optionally, combining the deferasirox salt solution obtained in step-(b) or step-(d) with a suitable metal salt to produce a reaction mass, wherein the metal salt is selected from the group consisting of organic and inorganic salts of magnesium, calcium and zinc; and
f) isolating and/or recovering the crystalline form of deferasirox salt either from the second solution obtained in step-(b) or from the third solution obtained in step-(d) or from the reaction mass obtained in step-(e).

6. The process of claim 5, wherein the first and second solvents used in steps-(a) & (d) are, each independently, selected from the group consisting of water, methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, N,N-dimethylformamide, N,N-dimethylacetamide, dimethylsulfoxide, and mixtures thereof; wherein the metal salt used in step-(e) is used in the form of an aqueous solution; wherein the organic salt used in step-(e) is a carboxylate salt or a sulfonate salt; and wherein the inorganic salt used in step-(e) is a halide salt, a borate salt, a phosphate salt or a sulfate salt.

7. (canceled)

8. The process of claim 6, wherein the first and second solvents are, each independently, selected from the group consisting of water, methanol, ethanol, isopropyl alcohol, n-butanol, tert-butanol, amyl alcohol, isoamyl alcohol, acetone, and mixtures thereof; and wherein the metal salt used in step-(e) is selected from the group consisting of magnesium chloride, magnesium bromide, magnesium iodide calcium chloride calcium bromide, calcium fluoride, calcium iodide, zinc chloride and zinc bromide.

9. (canceled)

10. The process of claim 5, wherein the first solution in step-(a) is prepared by dissolving deferasirox in the first solvent at a temperature below a boiling temperature of the solvent, and wherein the suspension in step-(a) is provided by suspending deferasirox in the first solvent while stirring at a temperature below boiling temperature of the first solvent; or wherein the first solution or suspension in step-(a) is prepared by reacting 2-(2-hydroxyphenyl)-4H-1,3-benzoxazin-4-one with 4-hydrazinobenzoic acid in a reaction inert solvent under suitable conditions to produce a reaction mass containing crude deferasirox, subjecting the reaction mass to washings, extractions or evaporations, and dissolving, suspending or extracting the resulting deferasirox in the first solvent at a temperature below boiling temperature of the first solvent.

11. The process of claim 10, wherein the deferasirox is dissolved in the first solvent at a temperature of about 25° C. to about 110° C.; and wherein the first solution or suspension obtained in step-(a) is optionally stirred at a temperature of about 30° C. to the reflux temperature of the first solvent for about 20 minutes to about 8 hours.

12-17. (canceled)

18. The process of claim 5, wherein the first solution obtained in step-(a) is optionally subjected to carbon treatment or silica gel treatment; wherein the base in step-(b) is used in a molar ratio of about 0.8 to 2.5 moles per mole of deferasirox free acid; wherein the combining in step-(b) is accomplished by adding the first solution or suspension to the base or by adding the base to the first solution or suspension; wherein the second solution obtained in step-(b) is optionally subjected to carbon treatment or silica gel treatment; wherein the removal of the first solvent in step-(c) is accomplished by substantially complete evaporation of the first solvent, concentrating the solution or distillation of first solvent under inert atmosphere, or a combination thereof; wherein the residue containing deferasirox salt in step-(d) is dissolved in the second solvent at a temperature below about reflux temperature of the second solvent wherein the metal salt in step-(e) is used in a molar ratio of about 0.3 to 1.5 moles per mole of deferasirox free acid; wherein the combining in step-(e) is accomplished by adding the deferasirox salt solution to the metal salt or by adding the metal salt to the deferasirox salt solution; and wherein the isolation in step-(f) is carried out by cooling, seeding, partial removal of the solvent from the solution, by adding an anti-solvent to the solution, or a combination thereof.

19-24. (canceled)

25. The process of claim 18, wherein the base is used in a molar ratio of about 1.0 to 2.0 moles per mole of deferasirox free acid; wherein the addition in step-(b) is carried out at a temperature of about 15° C. to about 85° C.; wherein the reaction mass obtained after completion of the addition process in step-(b) is stirred at a temperature of about 40° C. to about 80° C. for about 30 minutes to about 8 hours to produce a second solution; wherein the distillation process in step-(c) is performed at atmospheric pressure or reduced pressure; wherein the distillation process in step-(c) is carried out at a temperature of about 30° C. to about 110° C.; wherein the residue in step-(d) is dissolved in the second solvent at a temperature of about 40° C. to about 80° C.; wherein the metal salt in step-(e) is used in a molar ratio of about 0.4 to 0.7 moles per mole of deferasirox free acid; wherein the addition in step-(e) is carried out at a temperature of about 30° C. to about 85° C.; and wherein the isolation in step-(f) is carried out by cooling the solution at about 0° C. to about 25° C. for about 30 minutes to about 20 hours.

26-53. (canceled)

54. The process of claim 5, wherein recovering in step-(f) is carried out by filtration, filtration under vacuum, decantation, centrifugation, filtration employing a filtration media of a silica gel or celite, or a combination thereof; wherein the substantially pure crystalline form of deferasirox salt obtained in step-(f) is further dried under vacuum or at atmospheric pressure, at a temperature of about 35° C. to about 70° C.; and wherein the crystalline form of deferasirox salt obtained in step-(f) has a purity of about 99% to about 99.95% as measured by HPLC.

55-58. (canceled)

59. The process of claim 5, wherein the crystalline forms of deferasirox salts are prepared as follows:
i) the crystalline deferasirox sodium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution; and
d) isolating crystalline deferasirox sodium from the solution;
ii) the crystalline deferasirox potassium salt is prepared by a process comprising:
a) providing a solution or a suspension of deferasirox in a solvent selected from the group consisting of water alcohols and mixtures thereof;
b) adding potassium hydroxide to the solution or suspension obtained in step-(a);
c) heating the reaction mass obtained in step-(b) to form a clear solution;
d) optionally, concentrating the solution obtained in step-(c);
e) optionally, dissolving the residue obtained in step-(d) in an alcoholic solvent; and
f) isolating crystalline deferasirox potassium salt from the solution obtained in step-(c) or step-(e);
iii) the crystalline deferasirox magnesium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution;
d) adding aqueous magnesium chloride solution;
e) optionally, filtering the mass obtained in step-(d) to remove any extraneous matter; and
f) isolating crystalline deferasirox magnesium from the solution;
iv) the crystalline deferasirox calcium salt is prepared by a process comprising:
a) providing a solution or a suspension of deferasirox in a solvent selected from the group consisting of water, alcohols, and mixtures thereof;
b) adding sodium hydroxide to the solution or suspension obtained in step-(a);
c) heating the reaction mass obtained in step-(b) to form a clear solution;
d) adding aqueous calcium chloride solution;
e) optionally, filtering the mass obtained in step-(d) to remove any extraneous matter; and
f) isolating crystalline deferasirox calcium from the solution;
v) the crystalline deferasirox zinc salt is prepared by a process comprising:
a) providing a solution or a suspension of deferasirox in a solvent selected from the group consisting of water alcohols and mixtures thereof;
b) adding sodium hydroxide to the solution or suspension obtained in step-(a);
c) heating the reaction mass obtained in step-(b) to form a clear solution;
d) adding aqueous zinc chloride solution;
e) optionally, filtering the mass obtained in step-(d) to remove any extraneous matter; and
f) isolating crystalline deferasirox zinc salt from the solution;
vi) the crystalline deferasirox triethylamine salt is prepared by a process comprising:
a) providing a solution of deferasirox in an alcoholic solvent;
b) adding triethylamine to the solution obtained in step-(a); and
c) isolating crystalline deferasirox triethylamine salt from the solution;
vii) the crystalline deferasirox dimethylamine salt is prepared by a process comprising:
a) providing a solution of deferasirox in an alcoholic solvent;
b) adding dimethylamine to the solution obtained in step-(a); and
c) isolating crystalline deferasirox dimethylamine salt from the solution;
viii) the crystalline deferasirox tert-butylamine salt is prepared by a process comprising:
a) providing a solution of deferasirox in an alcoholic solvent;
b) adding tert-butylamine to the solution obtained in step-(a); and
c) isolating crystalline deferasirox tert-butylamine salt from the solution.

60-66. (canceled)

67. A pharmaceutical composition comprising solid state form of a deferasirox salt and one or more pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.

68. The pharmaceutical composition of claim 67, wherein the pharmaceutical composition is a solid dosage form, an oral suspension, a liquid, a powder, an elixir, an aerosol, a syrup, or an injectable solution.

69. The pharmaceutical composition of claim 67, wherein the solid state form of deferasirox salt has a D₉₀ particle size of less than or equal to about 500 microns.

70. The pharmaceutical composition of claim 69, wherein the solid state form of deferasirox salt has a D₉₀ particle size of less than or equal to about 300 microns; less than or equal to about 100 microns; less than or equal to about 60 microns; or less than or equal to about 15 microns.

71. A method for treating a patient suffering from diseases caused by chronic iron overload due to blood transfusions, comprising administering a pharmaceutical composition that comprises the solid state form of deferasirox salt of claim 1 along with pharmaceutically acceptable excipients, wherein the salt of deferasirox is a triethylamine salt, a dimethylamine salt, a tert-butylamine salt, a sodium (Na⁺) salt, a potassium (K⁺) salt, a magnesium (Mg²⁺) salt, a calcium (Ca²⁺) salt or a zinc (Zn²⁺) salt.
